(19) United States
(12) Patent Application Publication
(10) Pub. No.: US 2013/0324595 A1
(43) Pub. Date: Dec. 5, 2013

(54) C-GLUCOSIDIC ELLAGITANNIN COMPOUNDS FOR USE FOR ALTERING THE SUPRAMOLECULAR ARRANGEMENT OF ACTIN AND FOR THE TREATMENT OF OSTEOPOROSIS, CANCER, BACTERIAL INFECTION AND VIRAL INFECTION

(71) Applicants: Stephane Quideau, Pessac (FR); Elisabeth Genot, Pessac (FR); Frederic Saltel, Pessac (FR); Celine Douat, Pessac (FR); Daniela Melanie Delannoy Lopez, Pessac (FR)

(72) Inventors: Stephane Quideau, Pessac (FR); Elisabeth Genot, Pessac (FR); Frederic Saltel, Pessac (FR); Celine Douat, Pessac (FR); Daniela Melanie Delannoy Lopez, Pessac (FR)

(73) Assignee: INSTITUT NATIONAL DE LA SANTE ET DE LA RECHERCHE MEDICALE (INSERM), Paris (FR)

(21) Appl. No.: 14/000,766

(22) PCT Filed: Feb. 22, 2012
(86) PCT No.: PCT/EP2012/053017

§ 371 Date: Aug. 21, 2013

(30) Foreign Application Priority Data
Feb. 22, 2011 (EP) 11305186.6

(51) Int. Cl. A61K 31/365

(52) U.S. Cl. CPC A61K 31/365
USPC 514/450; 435/7.1

(57) ABSTRACT

The present invention concerns a C-glucosidic ellagitannin compound or a metabolite thereof for use for altering the supramolecular arrangement of actin in an individual suffering from osteoporosis, cancer, bacterial infection, or viral infection. It also pertains to pharmaceutical compositions comprising a C-glucosidic ellagitannin compound and/or metabolites thereof and one or more physiologically acceptable carriers. It finally concerns a C-glucosidic ellagitannin compound or a metabolite thereof, optionally detectably labeled, for in vitro use as a tool for studying cellular mechanisms involving actin, or for detecting F-actin in a cell.

The present invention concerns a C-glucosidic ellagitannin compound, or a metabolite, for use for altering the supramolecular arrangement of actin in an individual in need thereof. It also pertains to pharmaceutical compositions comprising a C-glucosidic ellagitannin compound and/or metabolites thereof and one or more physiologically acceptable carriers. It finally concerns a C-glucosidic ellagitannin compound or a metabolite thereof, optionally detectably labeled, for in vitro use as a tool for studying cellular mechanisms involving actin, or for detecting F-actin in a cell.

### BACKGROUND OF THE INVENTION

The Actin Cytoskeleton and Cancer

Cancer is an unregulated proliferation of cells due to loss of normal controls, resulting in unregulated growth, lack of differentiation, local tissue invasion, and often, metastasis. Cancer can develop in any tissue or organ at any age. Many cancers are curable if detected at an early stage, and long-term remission is often possible in later stages. However, cure is not always possible and is not attempted in some advanced cases. The development of drugs effective against cancer and having limited toxic side effects thus remains a critical need.

As a tumor grows, nutrients are provided by direct diffusion from the circulation. As tumor volume increases, tumor angiogenesis factors are produced to promote formation of the vascular supply required for further tumor growth.

Almost from inception, a tumor may shed cells into the circulation. Although most circulating tumor cells die as a result of intravascular trauma, an occasional cell may adhere to the vascular endothelium and penetrate into surrounding tissues, generating independent tumors (metastases) at distant sites. Metastatic tumors grow in much the same manner as primary tumors and may subsequently give rise to other metastases.

Experiments suggest that through random mutation, a subset of cells in the primary tumor may acquire the ability to invade and migrate to distant sites, resulting in metastasis.

In non cancer cells, adhesion to the extracellular matrix and to neighbouring cells plays a central role in the control of cell survival, growth, differentiation, motility, and tissue integrity. Upon oncogenic transformation, profound changes occur in the organization of the actin cytoskeleton, manifesting on cell morphology and motility. Increased proliferation, a hallmark of cancer cells, is highly dependent upon actin dynamics and cell adhesion. Adhesive interactions involve specialized transmembrane receptors that are linked to the cytoskeleton through junctional plaque proteins. The synthesis of several actin-binding proteins, including α-actinin, vinculin, tropomyosin and profilin, is down-regulated in transformed cells and overexpressing these proteins in tumor cells suppresses the transformed phenotype, which allows them to be considered as tumor suppressors.

The Actin Cytoskeleton and Osteoporosis

Osteoporosis is a progressive metabolic bone disease that decreases bone density with deterioration of bone structure. Skeletal weakness leads to fractures with minor or inapparent trauma, particularly in the thoracic and lumbar spine, wrist, and hip. Acute or chronic back pain is common. Prevention and treatment involve calcium and vitamin D supplements, exercises to maximize bone and muscle strength and minimize the risk of falls, and drug therapy to preserve bone mass or stimulate new bone formation.

Normally, bone formation and resorption are closely coupled. Osteoblasts (cells that make the organic matrix of bone and then mineralize bone) and osteoclasts (cells that resorb bone) are regulated by parathyroid hormone (PTH), calcitonin, estrogen, vitamin D, various cytokines, and other local factors such as prostaglandins.

Peak bone mass in men and women occurs by the mid 20s. Bone mass plateaus for about 10 yr, during which time bone formation approximately equals bone resorption. After this, bone loss occurs at a rate of about 0.3 to 0.5% per year. Beginning with menopause, bone loss accelerates in women to about 3 to 5% per year for about 5 to 7 year.

The major mechanism is increased bone resorption, which results in decreased bone mass and microarchitectural deterioration, even though other mechanisms also contribute to osteoporosis. The mechanisms of bone loss may involve local changes in the production of bone-resorbing cytokines (such as increases in cytokines that stimulate bone resorption), impaired formation response during bone remodeling (probably caused by age-related decline in the number and activity of osteoblasts), and other factors such as a decline in local and systemic growth factors.

The goals of treatment against osteoporosis are to preserve bone mass, prevent fractures, decrease pain, and maintain function. The rate of bone loss can be slowed with drugs (e.g. bisphosphonates or other anti-resorptive drugs) and, when possible, modification of risk factors. Calcium and vitamin D intake and physical activity must be adequate for drug treatment to be effective.

Bisphosphonates are first-line drug therapy. By inhibiting bone resorption, bisphosphonates preserve bone mass and can decrease vertebral and hip fractures by 50%. All increase bone mineral density and decrease risk of at least vertebral fractures. However, osteonecrosis of the jaw has been associated with use of bisphosphonates. Risk factors also include bisphosphonate use and cancer. Bisphosphonates may further be associated with atrial fibrillation, but the mechanism is not clear and there has been no association with increased cardiovascular mortality.

Estrogen can preserve bone density and prevent fractures. Most effective if started within 4 to 6 yr of menopause, estrogen may slow bone loss and possibly reduce fractures even when started much later. However, use of estrogen increases the risk of thromboembolism and endometrial cancer and may increase the risk of breast cancer. The risk of endometrial cancer can be reduced in women with an intact uterus by taking a progestin with estrogen. However, taking a combination of a progestin and estrogen increases the risk of breast cancer, coronary artery disease, stroke, and biliary disease.

PTH, which stimulates new bone formation, is generally reserved for patients who cannot tolerate anti-resorptive drugs or have contraindications to their use, or fail to respond to anti-resorptive drugs, as well as calcium, vitamin D, and exercise, developing new fractures and loss of bone mineral density, or possibly have severe osteoporosis.

Thus, the development of a treatment effective against osteoporosis and having limited side effects remains a critical need.

The osteoclast is the specialized cell that is responsible for bone resorption. It is a highly polarized cell that must adhere to the bone surface, where it undergoes alternative cycles of migration and resorption. Actin reorganization is critical for both processes. Osteoclast motility is mediated by podosomes, which are highly dynamic F-actin structures. Resorbing osteoclasts form a related actin complex, the sealing zone, which provides the boundary for the resorptive microenvironment. Similar to podosomes, the sealing zone is highly dependent on actin dynamics to allow efficient resorption. The integrity of the supramolecular arrangement of actin also plays a major role in the formation of the osteoclastic actin ring, a prerequisite for bone resorption.

In 1995, Hatano et al. have identified a new tannin, camelliatannin D, which potentially inhibits bone resorption by inhibiting calcium release, and which could be used as a treatment of osteoporosis (Chem Pharm Bull. 1995; 43(11):2033-5).

In 2004, Park et al. have demonstrated that a hydrolysable tannin, furosin, has an inhibitory effect on osteoclast differentiation and function, probably by inhibiting the early stage of osteoclastic differentiation and the actin ring formation (Biochem Biophys Res Commun. 2004; 325(4):1472-80).

In 2006, Hoffmann et al. have shown that naturally occurring ellagitannins inhibit osteoclast differentiation and function and induce apoptosis in osteoblasts, with roburin C and D having the highest potency. This descriptive study showed, on the one hand, that the osteoclast actin ring formation was reduced and, on the other hand, that bone resorption was significantly affected by the presence of the most potent ellagitannins. No evidence of any interaction of any kind between the tested ellagitannins and the actin ring was brought by the authors (Bone. 2006; 39(5):S3-40).

### DESCRIPTION OF THE INVENTION

The ellagitannins are a class of hydrolysable tannins formed when gallic acid, a phenol monomer, esterifies with the hydroxyl groups of a polyol carbohydrate such as glucose and oxidatively couples into hexahydroxydiphenoyl (HHDP) units, from which ellagic acid can be hydrolytically released. As used herein, the expression “C-glucosidic ellagitannins” refers to a particular group of ellagitannins, essentially occurring in plant species of only three subclasses of the Cronquist angiosperm classification (i.e., Hamamelidae, Rosidae and Dilleniidae), that comprises a very unique series of highly hydrosoluble C-glucosidic variants in that the usual glucopyranose core is replaced by a rarely encountered-in-nature open-chain glucose resulting from the establishment of their C-aryl glucosidic bond. Several of these C-glucosidic ellagitannins further display a nonahydroxyterphenoyl (NHTP) unit triply connected at positions 2, 3 and 5 of their glucose core (FIG. 1).

The inventors have described for the first time the effects of several C-glucosidic ellagitannin on cellular actin, one of the most abundant structural proteins in eukaryotic cells (Example 1). Monomeric globular actin (G-actin) subunits assemble via an ATP-dependent process into polymeric fibrillar actin (F-actin) filaments that are further ordered into three-dimensional architectures by interacting with so-called actin-binding proteins (ABPs) to establish the functional actin cytoskeleton. As used herein, the expression “supramolecular arrangement of actin” refers to such three-dimensional architectures of polymeric F-actin filaments. A dynamic equilibrium between the G-actin and F-actin states continuously ensures the adaptation of the actin cytoskeleton during its various implications in determining and/or controlling inter alia cell shape, cytokinesis, motility, adhesion and gene expression.

The inventors have surprisingly found that the C-glucosidic ellagitannin vescalagin interacts with F-actin, and alters the supramolecular arrangement of actin by winding the actin filaments into fibrillar aggregates (Examples 5 and 6).

The inventors have also found that the C-glucosidic ellagitannin vescalagin is capable of crossing the plasma membrane, and that it specifically binds polymeric F-actin, thus interfering with its function, but not with micro-tubules. Furthermore, they have found that it does not induce depolymerization of F-actin, but rather promotes the polymerization of F-actin, thus displacing the G-actin/F-actin equilibrium in favour of F-actin. They have also shown that the actin cytoskeleton C-glucosidic ellagitannins-induced alterations were reversible by washing after a treatment of 1 hour with 100 μM vescalagin and 24 hours with 50 μM vescalagin, underlining the limited toxicity of these compounds when used at reasonable doses and for a limited time. Finally, they have found that these compounds do not bind the same site in F-actin as phalloidin, a molecule known for binding filamentous actin.

Therefore, a first aspect of the invention is a C-glucosidic ellagitannin compound and/or a metabolite thereof for use for altering the supramolecular arrangement of F-actin in an individual in need thereof.

The C-glucosidic ellagitannins consist of vescalagin, vescalin, castalagin, castalin, grandinin, roburin A, roburin B, roburin C, roburin D, and roburin E. Preferably, the compound for use for altering the supramolecular arrangement of actin in an individual in need thereof is selected from the group consisting of vescalagin (CAS 36001-47-5), vescalin, castalagin (CAS 24312-00-3), and castalin (CAS 19086-75-0), and metabolites thereof.

The metabolite of the C-glucosidic ellagitannin according to the invention may refer to any intermediate or product of the C-glucosidic ellagitannin metabolism, such as:
ellagic acid, which may be derived from the hydrolysis of native C-glucosidic ellagitannins such as vescalagin, castalagin, grandinin and roburins A-E,
dimethyl ellagic acid,
urolithin A, urolithin B, urolithin C, urolithin D, which may notably be derived from gradual metabolism of ellagic acid by the intestinal microbiota, or
conjugates thereof such as glucosides or glucuronides of urolithin A, of urolithin B, of urolithin C, of urolithin D, of ellagic acid or of dimethyl ellagic acid.

The C-glucosidic ellagitannins or the metabolites thereof according to the invention may be obtained by extraction from oak or chestnut wood. Besides, vescalin and castalin may also be produced by hemi-synthesis from vescalagin and from castalagin, respectively, in particular with concomitant production of ellagic acid, as described by the inventors in Quideau et al., Chem. Eur. J. 2005, 11, 6503-6513.

By “altering/alteration of the supramolecular arrangement of actin” is meant any kind of modification of the supramolecular arrangement of actin. The supramolecular arrangement of actin may for instance be disrupted. The “alteration of the supramolecular arrangement of actin” may for instance correspond to a collapse of actin filaments and/or of actin stress fibers. It may also correspond to a stabilization of F-actin. It may further correspond to a cross-linking of F-actin “Altering of the supramolecular arrangement of actin” may also mean enhancing the assembly of monomeric globular actin (G-actin) subunits into polymeric fibrillar actin (F-actin) filaments, or shifting the dynamic equilibrium between the G-actin and F-actin states towards F-actin. “Altering of the supramolecular arrangement of actin” may alternatively mean interfering with the formation of the actin three-dimensional architecture, for instance by altering the interaction with actin-binding proteins (ABPs).

“An individual in need thereof” refers to an individual suffering from any disease or from any affection, the mechanism of which progression and/or onset implies the integrity of the supramolecular arrangement of actin. The individual to be treated in the frame of the invention is preferably a mammal, human or non human. It may also be a rodent, a feline, a canine, a bovine, an equine or an ovine. Preferably, the individual to be treated is a human being.

The integrity of the supramolecular arrangement of actin is required for most of the actin functions such as controlling cell shape, cytokinesis, cell motility, cell adhesion and gene expression.

Many pathogens, such e.g. viruses or bacteria, use these functions to spread from cell to cell in the body. Altering the supramolecular arrangement of actin may thus prevent pathogen spread in the body. Therefore, “an individual in need thereof” may refer to an individual suffering from a viral or bacterial infection.

The integrity of the supramolecular arrangement of actin is also required for the osteoclasts function. The osteoclast is the specialized cell that is responsible for bone resorption. It is a highly polarized cell that must adhere to the bone surface, where it undergoes alternative cycles of migration and resorption. Actin reorganization is critical for both processes. Osteoclast motility is mediated by podosomes, which are highly dynamic F-actin structures. Resorbing osteoclasts form a related actin complex, the sealing zone, which provides the boundary for the resorptive microenvironment. Similar to podosomes, the sealing zone is highly dependent on actin dynamics to allow efficient resorption. The integrity of the supramolecular arrangement of actin also plays a major role in the formation of the osteoclastic actin ring, a prerequisite for bone resorption. Altering the supramolecular arrangement of actin may prevent bone resorption and thus prevent or treat osteoporosis. Therefore, in a preferred embodiment, “an individual in need thereof” may refer to an individual suffering from osteoporosis.

As used herein, the term “osteoporosis” refers to any bone disease that decreases bone density and/or deteriorates bone structure. When the C-glucosidic ellagitannin compound used for altering the supramolecular arrangement of actin in an individual suffering from osteoporosis, compound preferably alters the osteoclastic actin ring, the podosome, and/or the sealing zone. Alteration of the osteoclastic actin ring, the podosome, and/or the sealing zone may be shown by various assays well known by the skilled in the art. For instance, alteration of the osteoclastic actin ring, the podosome, and/or the sealing zone may be visualized by fluorescence microscopy using e.g. phalloidin conjugated to FITC (Sigma) or Lifeact-mGFP to label F-actin. Such an alteration may be visualized by electron microscopy using immuno-gold-labeling of actin.

Actin functions such as controlling cytokinesis, cell motility, and cell adhesion are also essential for the development of cancer and for the apparition of metastasis. Therefore, altering the supramolecular arrangement of actin may also prevent the apparition and/or development of cancer tumors. Thus, in a specific embodiment, “an individual in need thereof” refers to an individual suffering from cancer and/or metastasis.

As used herein, the term “cancer” refers to any type of malignant (i.e. non benign) tumor. The malignant tumor may correspond to a primary tumor or to a secondary tumor (i.e. a metastasis). Further, the tumor may correspond to a solid malignant tumor, which includes e.g. carcinomas, adenocarcinomas, sarcomas, melanomas, mesotheliomas, blastomas, or to a blood cancer such as leukaemias, lymphomas and myelomas. According to an embodiment, the cancer is not a bone tumor or cancer. In a preferred embodiment, the cancer is a hyperproliferative and/or an invasive cancer. As used herein, “a hyperproliferative cancer” refers to a fast growing cancer comprising cells that have escaped apoptosis and show a high metabolic rate, and “an invasive cancer” refers to a cancer that comprises one or more secondary tumors or metastases.

The inventors have surprisingly found that treatment with the C-glucosidic ellagitannin vescalagin affects cellular morphology. Upon vescalagin treatment, cells have a more retracted appearance and exhibit irregular wound edges and retraction fibers, indicative of cell contraction (see Example 2). Thus, in a preferred embodiment, the C-glucosidic ellagitannin compound for use for altering the supramolecular arrangement of actin in an individual suffering from cancer inhibits cell adhesion and/or cell migration. The inhibition of cell adhesion and/or cell migration may be tested by various assays well known by the skilled in the art. For instance, the inhibition of cell adhesion and/or cell migration may be tested by scoring cell adhesion over time, by tracking cells under a videomicroscope, by using the wound assay or by using a transwell assay.

Vescalagin treatment also affects cellular viability: the inventors have shown that vescalagin treatment at 100 μM for 24 hours leads to irreversible commitment to cell death (see Example 2). Therefore, in a preferred embodiment, the C-glucosidic ellagitannin compound for use for altering the supramolecular arrangement of actin in an individual suffering from cancer induces cell death. As used herein, the expression “cell death” may refer to any kind of cellular death such as e.g. apoptosis or necrosis. Apoptosis and necrosis may for instance be quantified by several assays well-known by the skilled in the art such as e.g. annexin V staining. Apoptosis may also be specifically quantified by TUNEL assay, apoptosis-DNA ladder assay, appearance of pro-apototic markers or disappearance of anti-apoptotic markers and necrosis may for instance be discriminated from apoptosis by Propidium Iodide (PI) staining, a high PI staining being representative of necrosis.

Pharmaceutical Compositions According to the Invention

The invention further pertains to a pharmaceutical composition comprising a C-glucosidic ellagitannin and one or more physiologically acceptable carriers.

Pharmaceutical compositions comprising a C-glucosidic ellagitannin of the invention include all compositions wherein the C-glucosidic ellagitannin is contained in an amount effective to achieve the intended purpose. In addition, the pharmaceutical compositions may contain suitable physiologically acceptable carriers comprising excipients and auxiliaries which facilitate processing of the active compounds into preparations which can be used pharmaceutically.

The term “physiologically acceptable carrier” is meant to encompass any carrier, which does not interfere with the effectiveness of the biological activity of the active ingredient and that is not toxic to the host to which is administered. Suitable physiologically acceptable carriers are well known in the art and are described for example in Remington's Pharmaceutical Sciences (Mack Publishing Company, Easton, USA, 1985), which is a standard reference text in this field. For example, for parenteral administration, the above active ingredients may be formulated in unit dosage form for injection in vehicles such as saline, dextrose solution, serum albumin and Ringer's solution.

Besides the physiologically acceptable carrier, the compositions of the invention can also comprise minor amounts of additives, such as stabilizers, excipients, buffers and preservatives. The composition of the invention may further comprise a second active principle.

By “effective amount” is meant an amount sufficient to achieve a concentration of C-glucosidic ellagitannin which is capable of preventing, treating or slowing down the disease to be treated. Such concentrations can be routinely determined by those of skilled in the art. The amount of the C-glucosidic ellagitannin compound actually administered will typically be determined by a physician or a veterinarian, in the light of the relevant circumstances, including the condition to be treated, the chosen route of administration, the actual compound administered, the age, weight, and response of the subject, the severity of the subject's symptoms, and the like. It will also be appreciated by those of skilled in the art that the dosage may be dependent on the stability of the administered C-glucosidic ellagitannin.

Dosages to be administered depend on individual needs, on the desired effect and the chosen route of administration. It is understood that the dosage administered will be dependent upon the age, sex, health, and weight of the recipient, concurrent treatment, if any, frequency of treatment, and the nature of the effect desired. The total dose required for each treatment may be administered by multiple doses or in a single dose.

The C-glucosidic ellagitannin of the present invention may be administered by any means that achieve the intended purpose. For example, administration may be achieved by a number of different routes including, but not limited to subcutaneous, intravenous, intradermal, intramuscular, intraperitoneal, intracerebral, intrathecal, intranasal, oral, rectal, transdermal, buccal, topical, local, inhalant or subcutaneous use. Parenteral and topical routes are particularly preferred.

Depending on the intended route of delivery, the compounds may be formulated as liquid (e.g., solutions, suspensions), solid (e.g., pills, tablets, suppositories) or semisolid (e.g., creams, gels) forms.

In a preferred embodiment, the C-glucosidic ellagitannin composing the pharmaceutical composition is selected from the group consisting of vescalagin, vescalin, castalagin, castalin, grandinin, roburin A, roburin B, roburin C, roburin D, roburin E; still preferably from the group consisting of vescalagin, vescalin, castalagin, and castalin.

Molecular Delivery System According to the Invention

Depending on the intended route of delivery, the compound or the pharmaceutical composition of the invention may not be directly delivered on the site to be treated. Therefore, in a preferred embodiment, the compound or the pharmaceutical composition of the invention is in a mixture with a molecular delivery system.

As used herein, the expression “molecular delivery system” refers to any kind of system that increases the concentration of the compound or the pharmaceutical composition of the invention at the site to be treated. For instance, the site to be treated may be one or more tumors or one or more bones.

Mixing the compound or the pharmaceutical composition of the invention with a molecular delivery system may assure delivery to and maintenance at the site to be treated, leading to a better concentration of the compound at the site to be treated, and thus increasing the compound efficiency. Mixing the compound or the pharmaceutical composition of the invention with a molecular delivery system may also increase the compound solubility, protect the compound against degradation and/or reduce potential side effects or the compound or the pharmaceutical composition of the invention. Therefore, when the compound or the pharmaceutical composition of the invention is in a mixture with a molecular delivery system, said molecular delivery system preferably increases the compound solubility, maintains the compound on the site to be treated, protects the compound against degradation and/or increases the compound activity.

Beside allowing delivery to the site to be treated, said molecular delivery system may also allow controlling the time when the compound is delivered or not. For instance, the compound or the pharmaceutical composition of the invention may be delivered continuously during a certain period of time and then the delivery may be suspended for a certain period of time before being resumed. Alternatively, the compound or the pharmaceutical composition of the invention may be intermittently delivered. Thus, in a preferred embodiment, the molecular delivery system according to the invention allows spatio-temporal controlled delivery of the compound or of the pharmaceutical composition. In a specific embodiment, the molecular delivery system of the invention is BioChaperone™, a molecular delivery system commercialized by Adocia.

In Vitro Use of C-Glucosidic Ellagitannins or Derivates Thereof.

The inventors have surprisingly found that the C-glucosidic ellagitannins are capable of crossing the plasma membrane, and that they specifically bind polymeric F-actin, thus interfering with its function, but not with micro-tubules. Furthermore, they have found that vescalagin does not induce depolymerization of F-actin, but rather promotes the polymerization of F-actin, thus displacing the G-actin/F-actin equilibrium in favour of F-actin. They have also shown that the actin cytoskeleton vescalagin-induced alterations were reversible by washing after a treatment of 1 hour with 100 μM vescalagin and 24 hours with 50 μM vescalagin, underlining the limited toxicity of these compounds when used at reasonable doses and for a limited time. Finally, they have found that vescalagin does not bind the same site in F-actin as phalloidin, a molecule known for binding filamentous actin.

Therefore, vescalagin may be used as a tool for the study of the cytoskeleton. In particular, it may be used for investigating cytoskeleton structure and function, and the implication of actin in various biological processes, such e.g. cell motility, ruffling, cell division, contraction, cell morphology, cell stiffness and protein secretion.

Thus, another aspect of the invention is a C-glucosidic ellagitannin compound or a metabolite thereof, optionally detectably labeled, for in vitro use as a tool for studying cellular mechanisms involving actin. Also provided is in vitro use of a C-glucosidic ellagitannin compound or a metabolite thereof, optionally detectably labeled, as a tool for studying cellular mechanisms involving actin.

As used herein, “cellular mechanisms involving actin” may correspond to any cellular mechanism that may be impaired by disruption of the actin network integrity. The “cellular mechanisms involving actin” include for instance the control or regulation of cell shape, cytokinesis, cell motility, cell adhesion, gene expression and protein secretion.

The C-glucosidic ellagitannin compound or the metabolite thereof may also be used in vitro for inhibiting an interaction between F-actin and a compound liable to bind F-actin.

“A compound liable to bind F-actin” may be any compound that is capable of interacting with F-actin. Preferably, the compound liable to bind F-actin is a polypeptide. By “inhibiting an interaction” is meant preventing the binding of a molecule to another one. The inhibition of an interaction may be measured by various methods well-known by one skilled in the art. For instance, it may be measured by western blot assays, ELISA, co-immunoprecipitation (co-ip) assays, pull-down assays, crosslinking assays, label transfer approaches (FRET or HTRF assays) or yeast two-hybrid assays. The skilled in the art can easily determine if a compound inhibits an interaction between F-actin and a compound liable to bind to F-actin by carrying out a competitive binding assay.

Moreover, the present application discloses a method for synthesizing derivates of C-glucosidic ellagitannins, such as e.g. a vescalagin-FITC conjugate or a biotinylated vescalagin conjugate (see Example 4 and FIG. 3). They inventors have subsequently shown that these conjugate were able to bind actin and that the vescalagin-FITC conjugate highlighted the actin cytoskeleton, and in particular filamentous actin. Thus, in a specific embodiment, the C-glucosidic ellagitannin compound or a metabolite thereof for in vitro use for detecting F-actin in a cell may optionally be detectably labeled. The “detectably labeled compound” may for instance be conjugated to a fluorescent moiety such as e.g. fluorescein isothiocyanate (FITC), phycoerythrin (PE), allophycocyanin (APC), cyanin (Cy), Alexa Fluor (AF). The “detectably labeled compound” may also be a thiol derivative, a biotin conjugate or a radiolabelled variant or conjugate.

Vescalagin binding actin filaments, it may be a useful tool for investigating the distribution of F-actin in cells by labeling vescalagin with detectable label such as fluorescent moieties and using them to stain actin filaments for light microscopy. Fluorescent derivatives of vescalagin may be very useful in localizing actin filaments in living or fixed cells as well as for visualizing individual actin filaments in vitro. A high-resolution technique may developed to detect F-actin at the light and electron microscopic levels by using vescalagin conjugated to the fluorophore eosin which acts as a fluorescent tag. In this method known as fluorescence photo-oxidation, fluorescent molecules can be utilized to drive the oxidation of diaminobenzidine (DAB) to create a reaction product that can be rendered electron dense and detectable by electron microscopy. The amount of fluorescence visualized can be used as a quantitative measure of the amount of filamentous actin there is in cells if saturating quantities of fluorescent vescalagin are used. Consequently, immunofluorescence microscopy along with microinjection of vescalagin can be used to evaluate the direct and indirect functions of cytoplasmic actin in its different stages of polymer formation. Therefore, fluorescent vescalagin may be used as an important tool in the study of actin networks at high resolution.

Therefore, another aspect of the invention is a detectably labeled C-glucosidic ellagitannin compound or a metabolite thereof for in vitro use for detecting F-actin in a cell. Also provided is in vitro use of a detectably labeled C-glucosidic ellagitannin compound or a metabolite thereof for detecting F-actin in a cell.

The invention will be further illustrated in view of the following figures and examples.

### BRIEF DESCRIPTION OF THE FIGURES

FIG. 1. Structures of four oak-derived C-glucosidic ellagitannins.

FIG. 2. Live imaging of BAEc expressing actin-GFP were subjected to FRAP in the absence or presence of vescalagin (100 μM). A. A boxed region (200×100 pixel square) was photobleached [before (t 0″), immediately after (t 38″) and after photobleaching (t 98″)]; and normalized fluorescence intensity in the boxed region is shown for the entire duration of the FRAP experiment. Fluorescence recovery (starts at the red dot) recorded over time reveals the rates of actin turnover within this area. B. Quantitation of the results showing the immobile fraction as calculated from the difference between pre- and post-photobleaching intensities (n=6).

FIG. 3. Synthesis of the fluorescent vescalagin-FITC conjugate.

FIG. 4. A. Actin polymerization at its steady state in both permissive and non-permissive conditions was continued (30 min) in the presence of either alexa633-phalloidin or vescalagin-FITC. After high-speed centrifugation, F-actin stained with alexa633-phalloidin (blue) or with vescalagin-FITC (orange), but no F-actin was detected with FITC alone; b) when the fractions obtained under similar conditions were examined for actin content by SDS-PAGE, followed by Coomassie blue staining, cytochalasin D treatment yielded G-actin and F-actin in quantities similar to those obtained in the control (CT), whereas vescalagin- or phalloidin-treated samples showed a marked depletion of G-actin. B. This effect is dose-dependent. C. This effect is not altered by the presence of the FITC-bearing unit in vescalagin-FITC.

FIG. 5. Synthesis of the biotinylated vescalagin conjugate.

FIG. 6. SPR analysis of the binding of F-actin (top: curve a), G-actin (bottom: curve d), BSA (curve c) and streptavidin (curve b) to vescalagin: 217 RU of the biotinylated vescalagin conjugate were captured on a streptavidin-coated sensor chip. Solutions of each protein at 2 μM in running buffer were injected across the surface at 20 μmin for 147 seconds (first arrow) at 25° C. The dissociation phase (second arrow) was recorded for at least 300 seconds.

FIG. 7. Dose responses of endothelial cells following vescalagin or vescalin treatment. BAEc were incubated for 24 hours with TGF-β (which stimulates formation of podosomes), and different concentrations of vescalagin or vescalin were added either from the beginning of the TGF-β stimulation, or during the last hour of stimulation. Cells were then fixed and treated for immunofluorescence analysis. Podosomes were visualized by double staining using phalloidin (a F-actin marker) and cortactin (a podosome marker). The percentage of total cells showing podosomes was then quantified and compared to the control (cells incubated with TGF-β only). A. BAEc were stimulated for 24 hours with TGF-β, and vescalagin or vescalin was added during the last hour of stimulation. B. BAEc were stimulated for 24 hours with TGF-β, and vescalagin or vescalin was added from the beginning of the TGF-β stimulation.

### EXAMPLES

### Example 1

### C-Glucosidic Ellagitannins Induce Changes in Actin Configuration

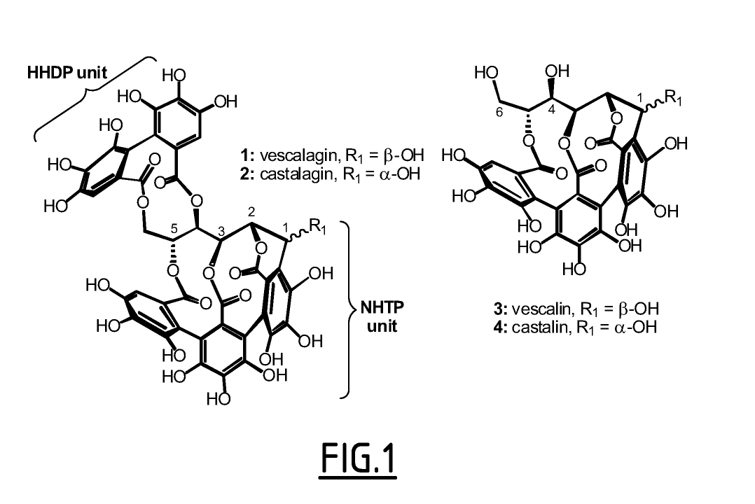

Vescalagin belongs to a particular group of ellagitannins, essentially occurring in plant species of only three subclasses of the Cronquist angiosperm classification (i.e., Hamamelidae, Rosidae and Dilleniidae), and which comprises a very unique series of highly hydrosoluble C-glucosidic variants in that the usual glucopyranose core is replaced by a rarely encountered-in-nature open-chain glucose resulting from the establishment of their C-aryl glucosidic bond. Another structural feature of several of these C-glucosidic ellagitannins, including vescalagin, is the presence of a nonahydroxyterphenoyl (NHTP) unit triply connected at positions 2, 3 and 5 of their glucose core (FIG. 1).

The inventors' initial interest in studying these C-glucosidic ellagitannins stems from the premise that the highly pre-organized medium-sized ring-containing multiple-phenol array featured by such natural products should be structurally well-suited to interfere with the construction of protein-made cellular architectures, on top of the list of which are actin filaments and microtubules. The inventors thus decided to probe the latter hypothesis by first examining the effect of a selection of four C-glucosidic ellagitannins on the actin cytoskeleton in living cells. Literature data on the capacity of some ellagitannins to interfere with bone resorption, which was notably accompanied by a disruption of osteoclastic actin rings, further backed the inventors' choice of selecting actin for this study. The selected compounds were the two most abundant C-glucosidic ellagitannins found in the heartwood of oak species, vescalagin and its C-1 epimer castalagin, and their corresponding two minor congeners, vescalin and castalin, both lacking the hexahydroxydiphenoyl (HHDP) unit at positions 4 and 6 of the glucose core (FIG. 1). The inventors used bovine aortic endothelial cells (BAEc), a well-characterized type of primary cells. Any of the four ellagitannins used at 50 μM rapidly provoked the disappearance of the internal stress fiber network observed in control cells. The inventors verified that cytochalasin D, known to inhibit F-actin polymerization, was also effective, yet, the perturbed actin configuration elicited by the four C-glucosidic ellagitannins appeared distinct from those induced by cytochalasin D. Focal adhesions, which anchor stress fibers to the matrix through integrins, underwent dissolution, suggestive of alterations in cell adhesion.

Overall, the four C-glucosidic ellagitannins elicited changes in actin configuration markedly different from those induced by other natural products known to target actin. The inventors thereafter focused on vescalagin, because it is the easiest to modify by (selective) synthetic chemical means, it is available in relatively large quantities by extraction from its main natural sources, and it moreover emerged as the most active compound of the series evaluated herein.

### Example 2

### Vescalagin Induces Rapid and Sustained Effects on Cellular Morphology

The vescalagin-induced F-actin disrupting effect seen in BAEc was also observed in fibroblast cells (baby hamster kidney cells, BHK), which also express β-actin as the main actin isoform, as well as in smooth muscle cells (A7r5), which in contrast predominantly express α-actin. Vescalagin induced similar collapse of F-actin bundles and cell contraction, but with varying potencies. Although subtle differences were noted among the three cell types tested, the impact of vescalagin on the actin cytoskeleton appeared neither cell- nor actin isoform-specific, suggesting that vescalagin can affect all types of mammalian cells. Furthermore, like in the case of cytochalasin D, no alteration of the microtubule network could be detected upon treating cells with vescalagin, indicating a specificity of this ellagitannin for actin. Remarkably, all cytoskeletal alterations could be completely reversed by washing out vescalagin from the cells with some fresh medium within 1 h. Phenotype recovery was achieved at a rate similar to that observed for cytochalasin D.

Vescalagin-induced dissolution of stress fibers affected cellular morphology and, eventually, viability. Within the range of concentrations and incubation times applied, observations made at the light microscopic level showed that cells changed their morphology from a well spread to a more retracted appearance. The cells exhibited irregular wound edges and retraction fibers, indicative of cell contraction upon vescalagin treatment. Mitosis was still observed when using vescalagin at 50 μM, but became impaired at 100 μM. Remarkably, propidium iodide (PI) staining revealed no cytotoxicity after 6 h and 24 h at 50 μM, but the presence of apoptotic nuclei was eventually detected when cells were exposed to vescalagin at 100 μM for 24 h, indicating in this case irreversible commitment to cell death. Functional consequences on cell behavior translated into impaired endothelial cell wound repair capacity. The efficient stimulation of the healing of mechanically injured endothelium observed in response to serum was reduced by about half in the presence of vescalagin at 20 μM. The denudated area remained virtually unpopulated after 6 h of incubation in medium containing vescalagin at 100 μM, conditions which are still compatible with the maintenance of cell viability. Cytochalasin D produced similar effects on cell migration. Overall, this ensemble of experimental data illustrates the rapid and sustained effects of vescalagin on cell behavior.

### Example 3

### Vescalagin Affects Both Thin and Thick Actin Fibers

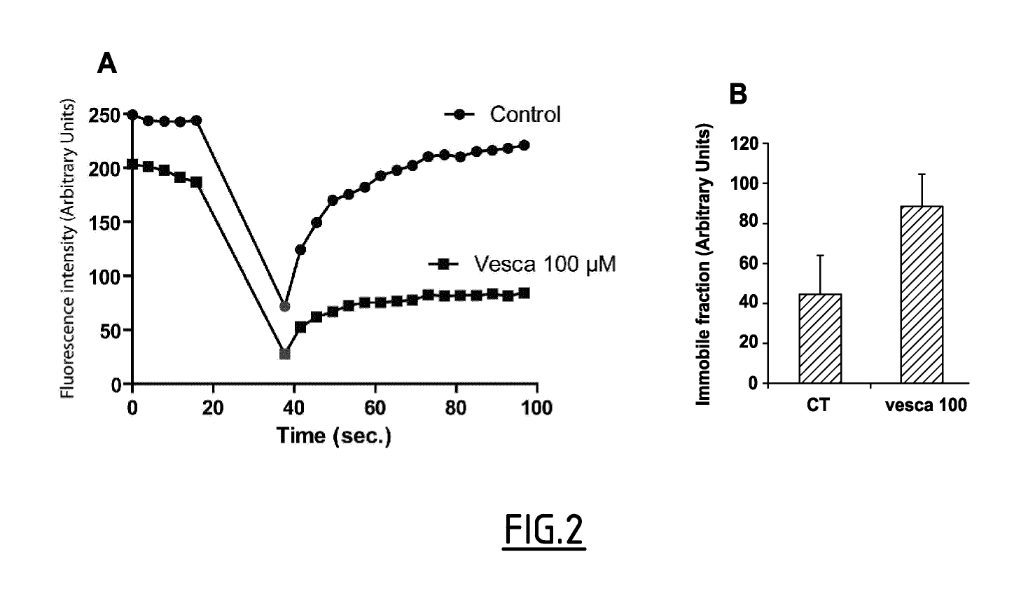

Live imaging carried out on BAEc expressing an actin-GFP construct and treated with vescalagin at 100 μM demonstrated immediate alteration of actin-GFP distribution at cell margin. Destabilization of the stress fibers was visualised by the progressive loss of filamentous staining, concomitantly with cell retraction as observed in phase contrast. Noticeably, thick F-actin bundles were maintained. To detect and unveil the internal dynamics of these apparently immobile F-actin bundles, the inventors performed a fluorescence recovery after photobleaching (FRAP) assay on actin-GFP expressing BAEc in the absence or presence of vescalagin (FIG. 2). The results indicate that vescalagin increases the immobile fraction of actin trapped into F-actin bundles and, therefore, also affects actin dynamics within these thick F-actin bundles. From these experiments, the inventors conclude that vescalagin affects both thin and thick actin fibers, F-actin bundles made of packed actin filaments being less vulnerable to the action of vescalagin than the single filament dendritic meshwork at the cell periphery.

### Example 4

### A Vescalagin-FITC Conjugate Directly Binds F-Actin

All together, these data demonstrate the rapidity with which vescalagin induces drastic perturbations within cells by collapsing most of the cytoskeleton architecture, strongly suggesting that, as in the case of others yet structurally different anti-actin drugs, vescalagin is capable of crossing the plasma membrane to target and destabilize actin. To investigate this further, the inventors relied on chemical synthesis to prepare a fluorescent vescalagin derivative and track it in cellulo. After having explored several possibilities of elaborating such a derivative by varying the nature of the fluorophore and its mode of attachment to the natural product using different types of linkers, the inventors settled on a vescalagin derivative equipped with a fluorescein-terminated 13-atom-long linker using fluorescein isothiocyanate (FITC) as the starting fluorophore. The choice of such a relatively long linker was made to prevent quenching of the fluorescein fluorescence through intramolecular interactions with the electronic-rich polyphenolic vescalagin-derived unit.

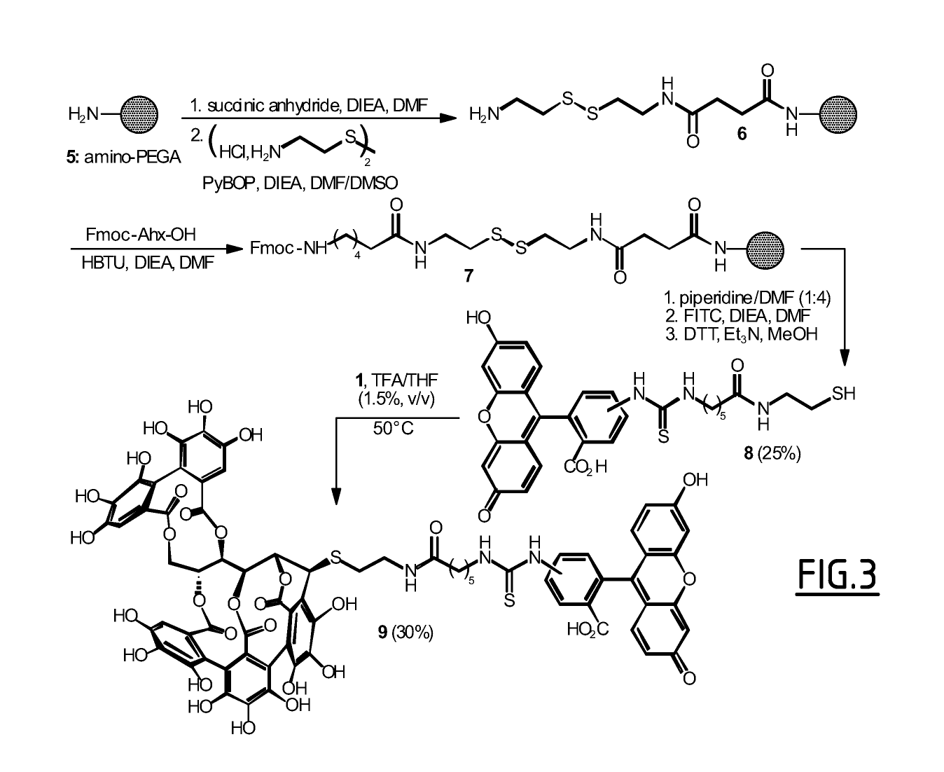

The synthesis of this vescalagin-FITC conjugate commenced with the preparation of the fluorescein-terminated linker on solid support (FIG. 3). An amino polyethylene glycol polyacrylamide (PEGA) copolymer-based resin was selected as the solid support, and was first treated with succinic anhydride in DMF at room temperature in the presence of N,N-diisopropylethyl amine (DIEA), then with cystamine dihydrochloride using (benzotriazole-1-yloxy)-tripyrrolidinophosphonium hexafluorophosphate (PyBOP) as coupling reagent to furnish the aminoethyl disulfide resin. Further extension of the tether of this resin was accomplished by reacting its primary amino group with N-Fmoc-6-aminohexanoic acid (Fmoc-Ahx-OH) using this time 2-(1H-benzotriazole-1-yl)-1,1,3,3-tetramethyluronium hexafluorophosphate (HBTU) as coupling reagent to furnish resin. The Fmoc protective group was removed under standard conditions to free the terminal amino group, which was then engaged in a final conjugation reaction with the isothiocyanate function of FITC (mixture of 5- and 6-fluorescein isothiocyanates). Release of the desired fluorescein-bearing thiourea-linked thiol from the solid support was conveniently achieved by using dithiothreitol (DTT) in methanol in the presence of Et₃N. Purification by reversed-phase HPLC furnished the fluorescein-bearing thiourea-linked thiol in an overall yield of 25%. With this thiol-functionalized fluorescein derivative in hands, the inventors took advantage of the remarkable chemoselectivity expressed at the vescalagin hydroxylated C-1 position under acid-catalyzed nucleophilic substitution reaction conditions to finally forge the vescalagin-FITC conjugate, which was purified by reversed-phase HPLC in 30% yield. This material was then tested for its biological activity. When added to living BAEc, the vescalagin-FITC conjugate displayed anti-actin effects similar to those observed for vescalagin. This fluorescent vescalagin derivative colocalised with F-actin in the remaining internal thick stress fibers and aggregates, thereby establishing that it has passed the plasma membrane and reached its target. To better visualise this target, BAEc were fixed to stabilize the actin cytoskeleton. After permeabilization, simultaneous staining with the vescalagin-FITC conjugate and fluorescent Alexa546-phalloidin revealed that, in the absence of the actin destabilisation effect, the vescalagin-FITC conjugate highlighted the entire cytoskeleton similarly to phalloidin, showing that the vescalagin-FITC conjugate binds all actin fibers in cellulo.

To explore whether or not the interaction between vescalagin and actin was direct, the inventors set up an in vitro assay based on actin polymerization from a solution of Ca²⁺-actin-ATP monomers. Spontaneous polymerization occurs when Ca²⁺ is replaced by Mg²⁺ provided by the F-actin buffer. The inventors performed high-speed centrifugation of the samples to separate the neo-formed polymers from the monomers to discriminate binding of vescalagin to either F-actin, G-actin or both. To visualize these presumptive molecular associations, the inventors used again the vescalagin-bearing fluoprobe and a fluorescent Alexa633-phalloidin to stain F-actin. Actin polymerization was carried out until it reached its steady state in both permissive and non-permissive conditions (i.e., in the presence or absence of Mg²⁺-containing F-buffer). The experiment was then continued for 30 min in the presence of either vescalagin-FITC conjugate or Alexa633-phalloidin, and the formation of actin polymers was then assessed by high-speed centrifugation. The results showed colored pellets consisting in insoluble actin stained with the fluorescent compound and indicated the expected binding of phalloidin onto filamentous actin (F-actin), as well as that of the vescalagin-FITC conjugate onto that insoluble actin material. Thus, the vescalagin-FITC conjugate directly binds F-actin.

### Example 5

### Vescalagin Binds to F-Actin and Promotes the Actin Filament State

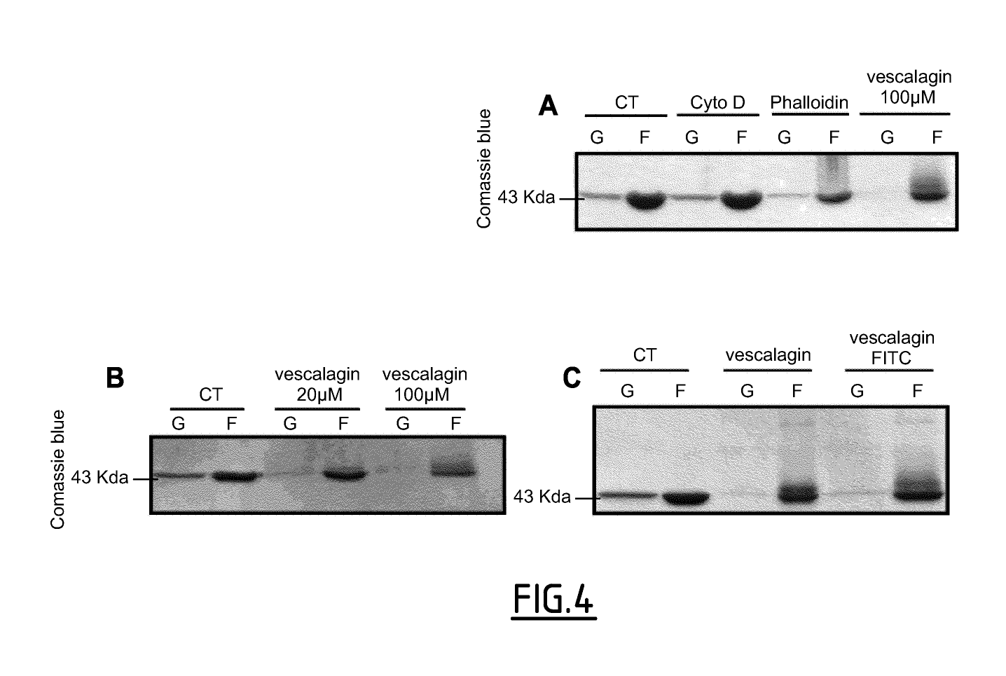

In the same way, quantifying the amount of F-actin (pellet) versus that of G-actin (supernatant) at the steady state was achieved by Western blot after high-speed centrifugation of samples treated with either vescalagin (100 μM), cytochalasin D (4 μM) or phalloidin (2 μM). Polymerization performed in the presence of cytochalasin D yielded a G-actin/F-actin ratio similar to that obtained in the control (FIG. 4A), as expected from the ability of this anti-actin drug to block actin assembly and disassembly, but vescalagin-treated samples were characterized by a marked depletion of G-actin, similar to that observed in the case of phalloidin-treated samples. Thus, it seems that vescalagin promotes the actin filament state, presumably by binding to F-actin and thereby displacing the equilibrium in favour of F-actin, in a dose-dependent manner (FIG. 4B). The same effect was observed when using the vescalagin-FITC conjugate (FIG. 4C).

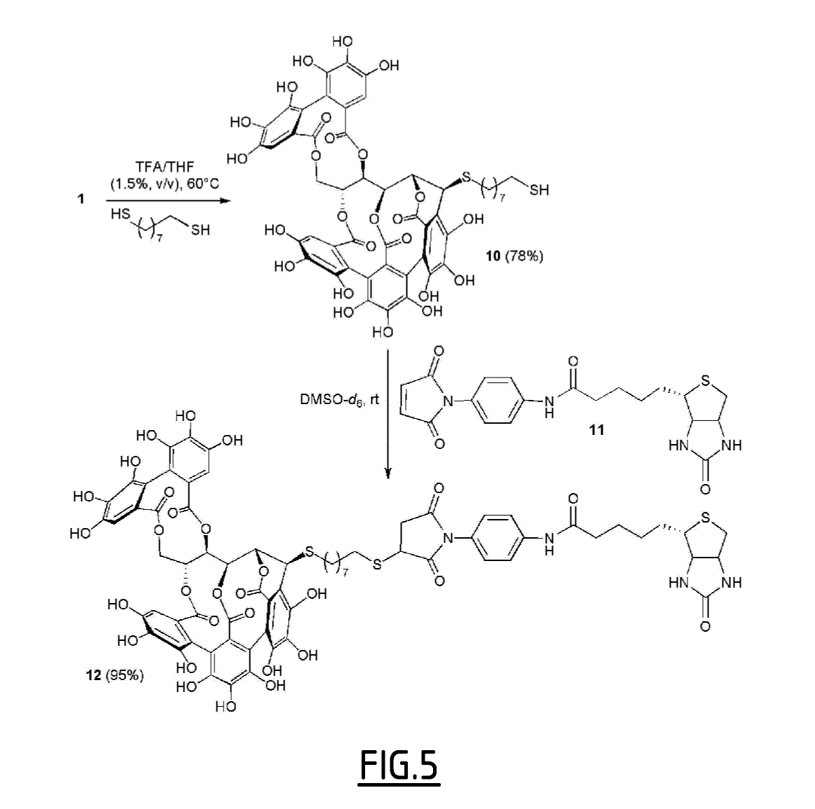

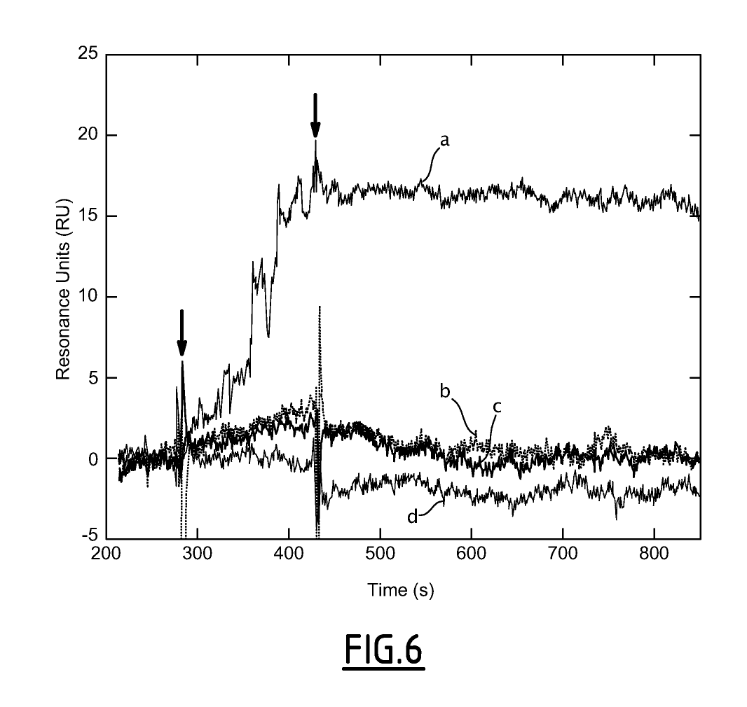

A surface plasmon resonance (SPR)-based analysis confirmed that vescalagin does not bind G-actin. A biotinylated vescalagin conjugate was synthesized by first reacting vescalagin with octane-1,8-dithiol to furnish the sulfhydryl thioether 1-deoxyvescalagin derivative (FIG. 5). This thiol was then coupled to the biotinylated maleimide linker. This coupling reaction was performed in deuterated DMSO to enable its monitoring by ¹H NMR spectroscopy. The reaction was complete after 7 h at room temperature and afforded pure biotinylated vescalagin conjugate in 95% yield by precipitating it from the reaction mixture upon addition of Et₂O-MeOH (3:1, v/v). This biotinylated vescalagin conjugate was then easily immobilized in one step on streptavidin-coated sensor chips to perform a series of SPR analyses using G-actin and F-actin (FIG. 6). No detectable binding occurred when G-actin was injected at a concentration of 2 μM over the vescalagin-coated surface. In contrast, when F-actin, prepared from a 2 μM G-actin solution, was injected over the same surface, the SPR response strongly increased, demonstrating that vescalagin only binds filamentous actin. The slow dissociation phase observed indicates that the vescalagin/F-actin complexes thus formed are highly stable. The absence of significant SPR responses upon injection of 2 μM bovine serum albumine (BSA) or streptavidin solutions further supports the specific nature of the interaction between vescalagin and F-actin.

### Example 6

### Vescalagin Aggregates Actin Filaments into Randomly Organized Clusters

How vescalagin affects actin polymerization was visualised by confocal microscopy. Actin polymerization was initiated with Alexa568-actin monomers in F-buffer. Live imaging showed free actin filaments undergoing changes of shape on a scale of seconds, elongating in all directions. At the steady state, the vescalagin-FITC conjugate was added. Instantly, actin filaments were seen agglutinating together into irregular modules, suggesting that vescalagin physically cross-linked F-actin. A merged image of actin (red) and vescalagin (green) fluorescences revealed uniform colocalization of actin aggregates with the vescalagin-FITC conjugate. Stabilization of filaments by phalloidin did not prevent this effect (data not shown). Therefore, vescalagin-induced actin filament collapse is not mediated by actin depolymerization. Importantly, when the experiment was performed using conditions under which actin remains monomeric (i.e., in the presence of CaCl₂-containing G-buffer), the vescalagin-FITC conjugate distribution appeared diffuse, confirming that vescalagin had no effect on G-actin. Furthermore, when Ca²⁺-actin was converted into Mg²⁺-actin using conditions under which actin still remains monomeric (i.e., in the presence of EGTA), the vescalagin-FITC conjugate was unable to aggregate actin, ruling out any contribution of the actin conformational change upon Ca²⁺/Mg²⁺ exchange prior to actin polymerization. However, when the vescalagin-FITC conjugate was added onto G-actin, actin aggregation was induced upon initiation of actin polymerization by addition of standard F-buffer, and sporadic clusters of aggregated F-actin were thereby visualized in multiple foci all over the coverslip. These clusters grew in size over time and, after 10 min, reached the appearance of aggregates. Similar results were obtained when the experiment was performed using vescalagin. 3D analysis of these clusters revealed a fibrillar arrangement of actin, but a random organization of these fibrils within these clusters. Collectively, these findings demonstrate that vescalagin becomes capable of binding actin only when polymerized or undergoing polymerization into filaments. Thus, vescalagin presents two functions; it binds F-actin and then winds filaments into balls.

The actin filament aggregation effect of vescalagin did not prevent actin polymerization and furthermore decreases the pool of G-actin. By extension, in cellulo, the spontaneous induction of disorganized aggregates of F-actin by vescalagin would be expected to circumvent regulated actin filament elongation at filament ends, leading to a cellular environment in which there is insufficient polymerization-competent G-actin to maintain normal stress fiber turnover. Alterations of cellular G-actin levels is known to regulate the synthesis of actin and of other actin regulatory proteins.

### Example 7

### Discussion

In conclusion, this work demonstrates that polyphenolic C-glucosidic ellagitannins constitute another pool of naturally occurring molecules that exert a privileged capacity for binding to actin. The ensemble of results the inventors gathered on vescalagin leads us to claim that it possesses all the requisites to be utilized as an anti-actin agent in cellular biological investigations under its natural form vescalagin or fluorescent version. It rapidly enters cells, and this despite its high hydrophilicity. Its dose-dependent effects on the actin cytoskeleton relies on its interaction with F-actin without any perturbation of the microtubule network. Like other anti-actin drugs but at variance with phalloidin, its effects on the actin cytoskeleton were found fully reversible, at least at the microscopic level within 1 h of treatment and at concentrations up to 100 μM, arguing for a non-covalent type of interaction with actin. The results found by the inventors are consistent with a mode of action through which the binding of vescalagin occurs along the length of the actin filament, probably at the protein-protein interface of the so-polymerized actin supramolecular association. The presence of two analogous polyhydroxylated arene motifs (i.e., the NHTP and HHDP units, see FIG. 1) likely constitutes the key structural feature that enables vescalagin to wind F-actin into balls by engaging it through multiple intra- and/or intermolecular contacts. Furthermore, since phalloidin retains its actin binding capacity for F-actin decorated with the vescalagin-FITC conjugate, as well as for vescalagin-induced actin aggregates, the inventors conclude that vescalagin does not bind at the same site(s) as phalloidin. Future studies will be devoted to the elucidation of the vescalagin-actin interaction at the molecular level and to the comparative evaluation of the anti-actin effects of the other C-glucosidic ellagitannins initially screened herein. Moreover, the SPR method the inventors developed using a biotinylated vescalagin conjugate to discriminate the binding of vescalagin to F- versus G-actin confirms the value of the technique for the study of polyphenol-protein interactions. Finally, the inventors have to recall that oak-derived C-glucosidic ellagitannins, including vescalagin, are present in wines as a result of their aging in oak-made barrels.

### Example 8

### Compared Dose Responses of Endothelial Cells Following Vescalagin or Vescalin Treatment

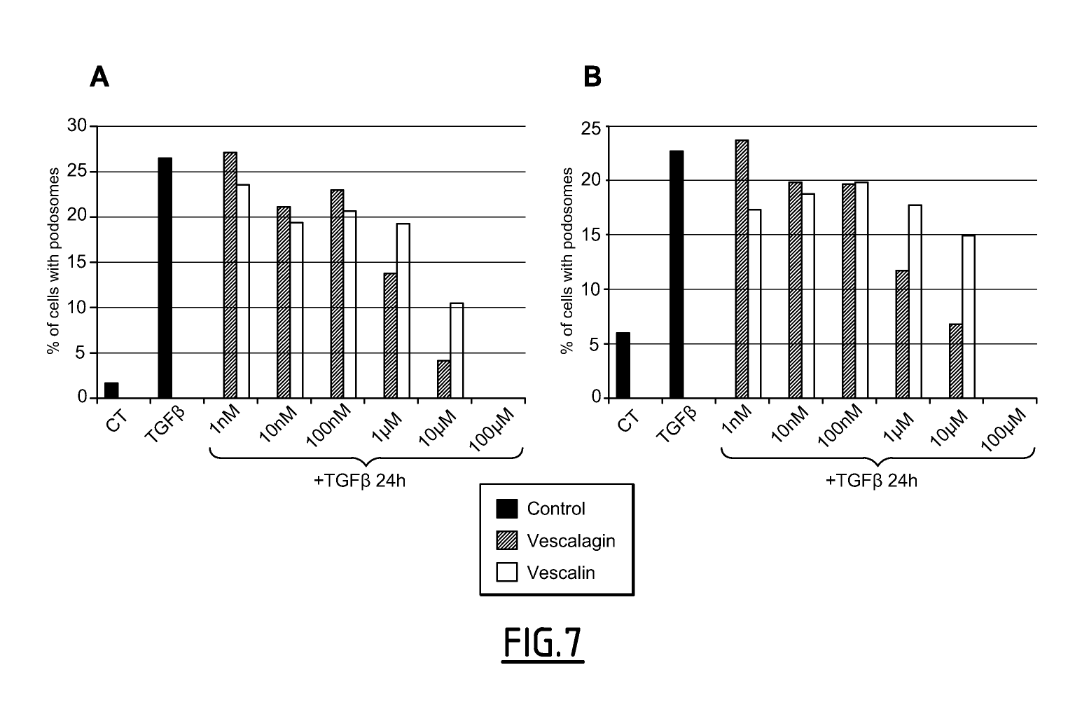

In this assay, bovine aortic endothelial cells (BAEc) were used. These cells form podosomes following a few hours of incubation with TGF-β. Cells were fixed after 24 hours of stimulation. Two protocols were tested. In the first protocol, cells were stimulated for 24 hours with TGF-β, and vescalagin or vescalin was added during the last hour of stimulation (FIG. 7A). In the second protocol, cells were stimulated for 24 hours with TGF-β, and vescalagin or vescalin was added from the beginning of the TGF-β stimulation (FIG. 7B). In both cases, cells were then fixed and treated for immunofluorescence analysis. Podosomes were visualized by double staining using phalloidin (a F-actin marker) and cortactin (a podosome marker). The percentage of total cells showing podosomes was then quantified and compared to the control (cells incubated with TGF-β only) (FIGS. 7A and 7B).

In this assay, vescalagin showed a stronger inhibitor activity than vescalin. The less pronounced effects observed when vescalagin or vescalin was added from the beginning of the TGF-β stimulation may be explained by rapid turn-over of these compounds.

1. (canceled)

2. The method of claim 16, wherein the compound is vescalagin or vescalin and the individual suffers from osteoporosis.

3. The method of claim 2, wherein the compound alters the osteoclastic actin ring, the podosome, or the sealing zone.

4. The method of claim 16, wherein the compound is a C-glucosidic ellagitannin compound, or a metabolite thereof selected from the group consisting of ellagic acid, dimethyl ellagic acid, urolithin A, urolithin B, urolithin C, urolithin D, and glucoside or glucuronide conjugates of urolithin A, of urolithin B, of urolithin C, of urolithin D, of ellagic acid or of dimethyl ellagic acid, and the individual suffers from cancer that is not bone cancer.

5. The method of claim 4, wherein the cancer is a hyperproliferative and/or an invasive cancer.

6. The method of claim 16, wherein the compound inhibits cell adhesion and/or cell migration.

7. The method of claim 16, wherein the compound induces cell death.

8. The method of claim 16, wherein the compound is selected from the group consisting of vescalagin, vescalin, castalagin, castalin, grandinin, roburin A, roburin B, roburin C, roburin D, roburin E, and metabolites thereof.

9. The method of claim 16, wherein the compound is selected from the group consisting of vescalagin, vescalin, castalagin, and castalin.

10. A pharmaceutical composition comprising a C-glucosidic ellagitannin compound and/or metabolites thereof and one or more physiologically acceptable carriers.

11. The pharmaceutical composition according to claim 10, wherein the C-glucosidic ellagitannin is selected from the group consisting of vescalagin, vescalin, castalagin, castalin, grandinin, roburin A, roburin B, roburin C, roburin D, roburin E and metabolites thereof.

12. The method of claim 16, wherein the compound is in a mixture with a molecular delivery system.

13. The method of claim 12, wherein the molecular delivery system increases the compound solubility, maintains the compound on the site to be treated, protects the compound against degradation and/or increases the compound activity.

14. An in vitro method for studying cellular mechanisms involving actin in a cell, comprising
treating said cell with a C-glucosidic ellagitannin compound or a metabolite thereof, optionally detectably labeled, and
determining whether said C-glucosidic ellagitannin compound or said metabolite thereof modulates cellular mechanisms involving actin in said cell.

15. (canceled)

16. A method of altering the supramolecular arrangement of actin in an individual in need thereof in order to treat osteoporosis, cancer that is not bone cancer, bacterial infection, or viral infection in said individual, comprising the step of
administering to said individual a C-glucosidic ellagitannin compound, or a metabolite thereof.

17. The pharmaceutical composition of claim 10, wherein the pharmaceutical composition is in a mixture with a molecular delivery system.

18. The pharmaceutical composition of claim 17, wherein the molecular delivery system increases the compound solubility, maintains the compound on the site to be treated, protects the compound against degradation and/or increases the compound activity.

19. An in vitro method of detecting F-actin in a cell, comprising
treating said cell with a detectably labeled C-glucosidic ellagitannin compound or a metabolite thereof and
detecting complexes of F-actin and said detectably labeled C-glucosidic ellagitannin compound or said metabolite thereof.
